SEQUENCE LISTING (25 sequences)

SEQ ID NO: 1 - protein, 9 aa, synthetic construct
GILGFVFTL

SEQ ID NO: 2 - protein, 9 aa, synthetic construct
  MOD_RES 1: note = Backbone C=O group replaced with a backbone C=S group
GILGFVFTL

SEQ ID NO: 3 - protein, 9 aa, synthetic construct
  MOD_RES 3: note = Backbone C=O group replaced with a backbone C=S group
GILGFVFTL

SEQ ID NO: 4 - protein, 9 aa, synthetic construct
  MOD_RES 4: note = Backbone C=O group replaced with a backbone C=S group
GILGFVFTL

SEQ ID NO: 5 - protein, 9 aa, synthetic construct
  MOD_RES 5: note = Backbone C=O group replaced with a backbone C=S group
GILGFVFTL

SEQ ID NO: 6 - protein, 9 aa, synthetic construct
  MOD_RES 6: note = Backbone C=O group replaced with a backbone C=S group
GILGFVFTL

SEQ ID NO: 7 - protein, 9 aa, synthetic construct
  MOD_RES 7: note = Backbone C=O group replaced with a backbone C=S group
GILGFVFTL

SEQ ID NO: 8 - protein, 9 aa, synthetic construct
  MOD_RES 8: note = Backbone C=O group replaced with a backbone C=S group
GILGFVFTL

SEQ ID NO: 9 - protein, 9 aa, synthetic construct
  MOD_RES 1: note = Backbone C=O group replaced with a backbone C=S group
  MOD_RES 8: note = Backbone C=O group replaced with a backbone C=S group
GILGFVFTL

SEQ ID NO: 10 - protein, 9 aa, synthetic construct
  MOD_RES 1: note = Backbone C=O group replaced with a backbone C=S group
  MOD_RES 4: note = Backbone C=O group replaced with a backbone C=S group
  MOD_RES 8: note = Backbone C=O group replaced with a backbone C=S group
GILGFVFTL

SEQ ID NO: 11 - protein, 9 aa, synthetic construct
  MOD_RES 1: note = Backbone C=O group replaced with a backbone C=S group
  MOD_RES 5: note = Backbone C=O group replaced with a backbone C=S group
  MOD_RES 8: note = Backbone C=O group replaced with a backbone C=S group
GILGFVFTL

SEQ ID NO: 12 - protein, 10 aa, synthetic construct
ELAGIGILTV

SEQ ID NO: 13 - protein, 10 aa, synthetic construct
  MOD_RES 1: note = Backbone C=O group replaced with a backbone C=S group
ELAGIGILTV

SEQ ID NO: 14 - protein, 10 aa, synthetic construct
  MOD_RES 3: note = Backbone C=O group replaced with a backbone C=S group
ELAGIGILTV

SEQ ID NO: 15 - protein, 10 aa, synthetic construct
  MOD_RES 4: note = Backbone C=O group replaced with a backbone C=S group
ELAGIGILTV

SEQ ID NO: 16 - protein, 10 aa, synthetic construct
  MOD_RES 5: note = Backbone C=O group replaced with a backbone C=S group
ELAGIGILTV

SEQ ID NO: 17 - protein, 10 aa, synthetic construct
  MOD_RES 6: note = Backbone C=O group replaced with a backbone C=S group
ELAGIGILTV

SEQ ID NO: 18 - protein, 10 aa, synthetic construct
  MOD_RES 7: note = Backbone C=O group replaced with a backbone C=S group
ELAGIGILTV

SEQ ID NO: 19 - protein, 10 aa, synthetic construct
  MOD_RES 8: note = Backbone C=O group replaced with a backbone C=S group
ELAGIGILTV

SEQ ID NO: 20 - protein, 10 aa, synthetic construct
  MOD_RES 9: note = Backbone C=O group replaced with a backbone C=S group
ELAGIGILTV

SEQ ID NO: 21 - protein, 10 aa, synthetic construct
  MOD_RES 1: note = Backbone C=O group replaced with a backbone C=S group
  MOD_RES 9: note = Backbone C=O group replaced with a backbone C=S group
ELAGIGILTV

SEQ ID NO: 22 - protein, 10 aa, synthetic construct
  MOD_RES 1: note = Backbone C=O group replaced with a backbone C=S group
  MOD_RES 6: note = Backbone C=O group replaced with a backbone C=S group
  MOD_RES 9: note = Backbone C=O group replaced with a backbone C=S group
ELAGIGILTV

SEQ ID NO: 23 - protein, 9 aa, synthetic construct
AAGIGILTV

SEQ ID NO: 24 - protein, 10 aa, synthetic construct
EAAGIGILTV

SEQ ID NO: 25 - protein, 10 aa, synthetic construct
FLPSDYFPSV

(19) United States
(12) Patent Application Publication
(10) Pub. No.: US 2025/0092087 A1
(43) Pub. Date: Mar. 20, 2025

(54) THIOAMIDE ANALOGUES OF PEPTIDE ANTIGENS

(71) Applicant: Wisconsin Alumni Research Foundation, Madison, WI (US)

(72) Inventors: Samuel Gellman, Madison, WI (US); Rylie Morris, Madison, WI (US); Ruslan Gibadullin, Brooklyn, NY (US); Jiani Niu, San Diego, CA (US)

(73) Assignee: Wisconsin Alumni Research Foundation, Madison, WI (US)

(21) Appl. No.: 18/887,770

(22) Filed: Sep. 17, 2024

Related U.S. Application Data
(60) Provisional application No. 63/583,804, filed on Sep. 19, 2023.

(51) Int. Cl. C07K 1/06; C07K 7/06

(52) U.S. Cl. CPC C07K 1/067; C07K 7/06

(57) ABSTRACT

A method to make polypeptide antigens with increased resistance to enzymatic degradation and the polypeptide antigens so formed. The method includes fabricating a polypeptide analogue of a native polypeptide antigen in which at least one backbone C═O group in the native polypeptide antigen residues is replaced with a backbone C═S group.

### CROSS-REFERENCE TO RELATED APPLICATIONS

Priority is hereby claimed to U.S. provisional application Ser. No. 63/583,804, filed Sep. 19, 2023, which is incorporated herein by reference.

### FEDERAL FUNDING STATEMENT

This invention was made with government support under GM056414 awarded by the National Institutes of Health. The government has certain rights in the invention.

### SEQUENCE LISTING

The instant application contains a Sequence Listing which has been submitted in an XML file with the USPTO through Patent Center and is hereby incorporated by reference in its entirety. The Sequence Listing XML, created on Jul. 23, 2024, is named “SEQ_LIST—09824545-P230415US02.xml” and is 34,105 bytes in size.

### BACKGROUND

CD8 T cell receptors (TCRs) recognize short peptide antigens, typically 8-10 residues, presented by a major histocompatibility complex I (MHC I) on the surface of an antigen-presenting cell. Interaction between the TCR and the peptide+MHC I complex (pMHC I) activates the T cell and triggers a cascade of responses that can lead to elimination of abnormal and infected cells. Synthetic peptides that correspond to MHC I epitopes are competent to induce antigen-specific CD8 T cell responses; such peptides have long been of interest as a basis for vaccine development. See, for example, Comber, J. D.; Philip, R. MHC Class I Antigen Presentation and Implications for Developing a New Generation of Therapeutic Vaccines. Ther Adv Vaccines 2014, 2 (3), 77-89. Extensive prior study has revealed a variety of factors, including the stability of pMHC I, that influence the potency of natural and modified antigen peptides in stimulating CD8 T cell activation. See, for example, Ekeruche-Makinde et al. Peptide Length Determines the Outcome of TCR/Peptide-MHCI Engagement. Blood 2013, 121 (7), 1112-1123. However, this approach to vaccine development has been hampered by the fact that short peptides comprised of proteinogenic L-α-amino acid residues are highly susceptible to cleavage by proteases.

Multiple strategies have been explored to suppress proteolytic susceptibility of MHC I peptides in vivo, such as replacement of one native L-α residue with a D-α- or β-amino acid residue, with a peptoid (N-alkyl-glycine) subunit, or multiple such replacements. See, for example, Cheloha, R. W.; Sullivan, J. A.; Wang, T.; Sand, J. M.; Sidney, J.; Sette, A.; Cook, M. E.; Suresh, M.; Gellman, S. H. Consequences of Periodic α-to-B3 Residue Replacement for Immunological Recognition of Peptide Epitopes. ACS Chem Biol 2015, 10 (3), 844-854, and Ballabio, et al. L- to d-Amino Acid Substitution in the Immunodominant LCMV-Derived Epitope Gp33 Highlights the Sensitivity of the TCR Recognition Mechanism for the MHC/Peptide Structure and Dynamics. ACS Omega 2022, 7 (11), 9622-9635. Although these approaches can be very effective for inhibiting protease action, both antigen MHC I affinity and TCR recognition of the pMHC I are very sensitive to structural alterations within the antigen. No general strategy has been identified for modifying peptide antigens in a manner that retains the necessary MHC I and TCR recognition properties while diminishing cleavage by proteases. Thus, there remains a long-recognized and unmet need for such a method.

### SUMMARY

Disclosed herein is a type of modification that has not previously been explored in the context of MHC antigen peptides: replacing a backbone C═O group with a backbone C═S group. The change creates a thioamide bond to the next adjacent residue, rather than an amide bond. This alteration is relatively subtle in terms of molecular structure. However, thioamide linkages between amino acid residues are less susceptible to protease action relative to the natural amide linkages. See, for example, Liu, et al. Fluorescent Probes for Studying Thioamide Positional Effects on Proteolysis Reveal Insight into Resistance to Cysteine Proteases. ChemBioChem 2019, 20 (16), 2059-2062. Key physical differences are: (1) a secondary thioamide is a stronger H-bond donor than the analogous amide; (2) the sulfur atom is a weaker H-bond acceptor than the oxygen atom; and (3) the C═S bond is longer than the corresponding C═O bond. Because of these physical differences, the C═O to C═S modification can influence the folding propensities of polypeptides. See, for example, Culik, R. M.; Jo, H.; Degrado, W. F.; Gai, F. Using Thioamides to Site-Specifically Interrogate the Dynamics of Hydrogen Bond Formation in β-Sheet Folding. J Am Chem Soc 2012, 134 (19), 8026-8029.

It is not possible to predict a priori whether or how replacing one or more backbone C═O groups with C═S groups might affect MHC I antigen properties. Using two well-studied antigens, one viral and the other cancer-associated, disclosed herein is a method of modifying antigens to make them more resistant to proteolytic degradation, without destroying their MHC I antigenic properties. Both antigens are restricted by the human leukocyte antigen-A2 (HLA-A2), an MHC I allele widely distributed in the human population. The method involves replacing backbone C═O units with C═S units, so that the link between two adjacent amino acid residues is converted from an amide group to a thioamide group. The resulting antigen analogues are designated “thio-antigens.” Initial efforts involved creating “thio-antigens” that contain a single thioamide unit (i.e., just one backbone C═O group was replaced with a C═S group); it was found that most of the singly-substituted analogues displayed strong HLA-A2 binding and evoked potent T cell responses, as measured by stimulation of secretion of interferon-γ (IFN-γ), a pro-inflammatory cytokine. See Hwang, J. R.; Byeon, Y.; Kim, D.; Park, S. G. Recent Insights of T Cell Receptor Mediated Signaling Pathways for T Cell Activation and Development. Experimental & Molecular Medicine 2020, 52 (5), 750-761. Guided by the data for these single replacement thio-antigens, disclosed herein are analogues containing one, two, three, or more dispersed backbone C═S groups in place of backbone C═O groups. These analogues were comparable to the natural antigen or more potent in terms of IFN-γ secretion. In addition, some thio-antigens with multiple substitutions displayed substantial resistance to cleavage by an aggressive and non-sequence selective protease (proteinase K). These findings indicate that thioamide derivatives of MHC I antigens provide a general basis for peptide vaccine development.

As noted, developing small polypeptides as vaccines that provide protective immunity is limited by rapid proteolytic degradation of the polypeptides. Introducing unnatural amino acid residues into these polypeptide antigens can suppress MHC I antigen proteolysis. However, the resulting modified peptides typically display lower affinity for the MHC I and/or diminished ability to activate CD8 T cells relative to the native antigen. Disclosed herein is a method for modifying MHC I antigens to enhance resistance to proteolysis while preserving MHC I affinity and T cell activation properties. This approach, replacing backbone C═O groups with backbone C═S, was evaluated in two well-characterized antigens presented by HLA-A2, a common human MHC I. For each antigen, singly-modified thioamide analogues retained affinity for HLA-A2 and activated T cells specific for the native antigen, as measured via IFN-γ secretion. In each system, a highly potent triply-substituted thioamide antigen (“thio-antigen”) was fabricated that displayed substantial resistance to proteolytic cleavage while retaining its antigenic properties.

Thus, disclosed herein is a method to make polypeptide antigens with increased resistance to enzymatic degradation. The method comprises fabricating a polypeptide analogue of a native polypeptide antigen in which at least one backbone C═O group in the native polypeptide antigen residues is replaced with a backbone C═S group, wherein the polypeptide analogue has an N-terminus and a C-terminus.

In one version of the method, at least two backbone C═O groups in the native polypeptide antigen residues are replaced with backbone C═S groups. One of the backbone C═S groups may be located at the N-terminus of the polypeptide analogue.

In a specific version of the method, none of the backbone C═S groups are located at the C-terminus of the polypeptide analogue. That is, the C-terminus amino acid residue remains as in the native polypeptide antigen, with a backbone C═O group.

In another version of the method, at least two or at least three backbone C═O groups in the native polypeptide antigen residues are replaced with backbone C═S groups. In a specific embodiment of this version of the method, only (and exactly) three backbone C═O groups in the native polypeptide antigen residues are replaced with backbone C═S groups.

It is generally preferred, although not required, that the native polypeptide antigen and the polypeptide analogue consist essentially of from 8 to 100 residues, or from 8 to 50 residues, or from 8 to 25 residues, or from 8 to 15 residues.

In yet another version of the method, at least three backbone C═O groups in the native polypeptide antigen residues are replaced with backbone C═S groups and none of the backbone C═S groups are located at the C-terminus of the polypeptide analogue (that is, the last residue of the polypeptide chain).

In any of the versions of the method disclosed herein, one option is that none of the backbone C═S groups are contiguous to one another. Also, it is optional that at least one of the backbone C═S groups is located at the N-terminus of the polypeptide analogue.

Also disclosed herein is a polypeptide antigen made by the method disclosed herein or having the molecular characteristics disclosed herein. That is, also disclosed herein is a non-natural polypeptide antigen with increased resistance to enzymatic degradation, the polypeptide antigen comprising from 8 to 100 residues linked by peptide bonds and having an N-terminus and a C-terminus, wherein at least one residues of the polypeptide antigen have a backbone C═O group replaced with a backbone C═S group, and wherein the residue at the C-terminus has a backbone C═O group. The polypeptide antigen may alternatively comprise from 8 to 50 residues, or from 8 to 25 residues, or from 8 to 15 residues.

The residue at the N-terminus may comprise a backbone C═S group. It is generally preferred, but not required that all the other remaining residues in the molecule (that is, those residues that do not contain a backbone C═S group) are α-amino acid residues, preferably L-α-amino acid residues. (This is not required. The remaining residues that do not contain a backbone C═S group may be, for example, beta-amino acid residues or other non-natural or non-proteinogenic amino acids.) The residues containing backbone C═S groups are preferably derived from L-α-amino acid residues.

Again, the disclosure encompasses molecules wherein at least two residues are residues in which a backbone C═O group is replaced with a backbone C═S group. The disclosure also encompasses molecules wherein at least three residues are residues in which a backbone C═O group is replaced with a backbone C═S group.

The objects and advantages of the disclosure will appear more fully from the following detailed description of the preferred embodiment of the disclosure made in conjunction with the accompanying drawings.

### BRIEF DESCRIPTION OF THE DRAWINGS

FIGS. 1A-1C. Structures and activities of thioamide-containing derivatives of the GIL antigen. (FIG. 1A) Sequences of GIL and analogues; sites of backbone C═O to C═S replacement are indicated by circled residues. (FIG. 1B) HLA-A2 stabilization on the surface of T2 antigen presenting cells by GIL and thioamide variants. Data points represent the average of ≥2 independent experiments. (FIG. 1C) Activation of GIL-specific CD8 T cells by GIL and thioamide variants, as measured by levels of secreted IFN-γ. Data points were generated from ≥2 independent experiments. All error bar uncertainties are expressed as S.E.M.

FIGS. 2A-2B. (FIG. 2A) Predicted cleavage sites of GILGFVFTL (1-9) (SEQ ID NO: 1; “GIL”) for proteinase K; prediction was performed by the PeptideCutter tool in the Expasy resource (web.expasy.org/peptide_cutter/) (Swiss Institute of Bioinformatics, Lausanne, Switzerland). Numbers indicate positions of predicted cleavage sites. (FIG. 2B) Proteolysis by proteinase K for GIL, GIL-9 and GIL-10 peptides evaluated in DPBS, pH 7.4. Peptide sequences are shown in FIG. 1A. Numbers above bars indicate peptide half-life (min).

FIGS. 3A-3C. Structures and activities of C═S-containing derivatives of the “ELA” antigen (ELAGIGILTV; SEQ ID NO:12). (FIG. 3A) Sequences of ELA and derivatives; circled residues indicate sites of C═O to C═S replacement. (FIG. 3B) Stabilization of HLAA2 on the surface of T2 cells by ELA and thioamide variants. Data points represent the average of ≥2 independent experiments. (FIG. 3C) Activation of ELA-specific CD8 T cells, as detected via IFN-γ production, by ELA and analogues. Data points were generated from ≥3 independent experiments. All error bar uncertainties are expressed as S.E.M.

FIGS. 4A-4B. (FIG. 4A) Prediction of cleavage sites for ELAGIGILTV (1-10) (SEQ ID NO: 12) epitope when treated with proteinase K, as calculated by the PeptideCutter tool in the Expasy resource. Numbers indicate positions of cleavage sites. (FIG. 4B) Proteolytic resistance against proteinase K for ELA, ELA-9 and ELA-10 peptides evaluated in DPBS, pH 7.4; peptide sequences are shown in FIG. 3A. Numbers above bars indicate peptide half-life (min).

FIGS. 5A-5B. (FIG. 5A) Side view of the ELAGIGILTV (1-10) (SEQ ID NO:12) antigen, shown as sticks in the HLA-A2 binding cleft (chain A residues 56-85 shown as ribbon in grey color) in the presence of MEL5 TCR (not shown); (PDB ID: 3HG1). (FIG. 5B) Side view of the GILGFVFTL (1-9) (SEQ ID NO:1) antigen, shown as sticks in the HLA-A2 binding cleft (chain A residues 56-85 shown as ribbon in grey color) in the presence of JM22 TCR (not shown); (PDB ID: 1OGA). Both figures were prepared using UCSF Chimera software (cgl.ucsf.edu/chimera/download.html) (Resource for Biocomputing, Visualization, and Informatics, San Diego, California).

FIG. 6. Pairwise HLA-A2 stabilization dose-response comparisons for the native GIL antigen and individual C═S-containing analogues displayed in FIG. 1B as a result of normalizing, averaging, and fitting median fluorescence data for live T2 cells to three-parameter sigmoidal curves. Data points represent the average of ≥2 independent experiments. All error bar uncertainties are expressed as S.E.M.

FIG. 7. Pairwise CD8 T cell activation (secretion of IFN-γ cytokine) dose-response comparisons for the native GIL antigen and individual C═S-containing analogues shown in FIG. 1C as a result of normalizing, averaging, and fitting optical density data to three-parameter sigmoidal curves. Data points were generated from ≥2 independent experiments. All error bar uncertainties are expressed as S.E.M.

FIG. 8. Proteolysis time course curves for GIL, GIL-9 and GIL-10 in the presence of proteinase K, as assessed by UPLC. Normalized data were fit to a one-phase decay model and represent the average of two (2) independent experiments. All uncertainties are expressed as S.E.M. Calculated peptide half-lives are shown in FIG. 2B.

FIG. 9. Pairwise HLA-A2 stabilization dose-response comparisons for the native ELA antigen and individual C═S-containing analogues displayed in FIG. 3B as a result of normalizing, averaging, and fitting median fluorescence data for live T2 cells to three-parameter sigmoidal curves. Data points represent the average of ≥2 independent experiments. All error bar uncertainties are expressed as S.E.M.

FIG. 10. Pairwise CD8 T cell activation (secretion of IFN-γ cytokine) dose-response comparisons for the native ELA antigen and individual C═S-containing analogues shown in FIG. 3C as a result of normalizing, averaging, and fitting optical density data to three-parameter sigmoidal curves. Data points were generated from ≥3 independent experiments. All error bar uncertainties are expressed as S.E.M.

FIG. 11. Proteolysis time course curves for ELA, ELA-9 and ELA-10 in the presence of proteinase K, as assessed by UPLC. Normalized data were fit to a one-phase decay model and represent the average of two (2) independent experiments. All uncertainties are expressed as S.E.M. Calculated peptide half-lives are shown in FIG. 4B.

### DETAILED DESCRIPTION

### Abbreviations and Definitions

All references to singular characteristics or limitations of the disclosed method shall include the corresponding plural characteristic or limitation, and vice-versa, unless otherwise specified or clearly implied to the contrary by the context in which the reference is made. The indefinite articles “a” and “an” mean “one or more.”

All combinations of method steps disclosed herein can be performed in any order, unless otherwise specified or clearly implied to the contrary by the context in which the referenced combination is made.

The method disclosed herein can comprise, consist of, or consist essentially of the essential elements and steps described herein, as well as any additional or optional ingredients, components, or limitations described herein or otherwise useful in organic chemistry. The disclosure provided herein suitably may be practiced in the absence of any element which is not specifically disclosed herein.

AcOH=acetic acid. DCM=dichloromethane. DIC=N,N′-diisopropylcarbodiimide. DIEA=N,N-diisopropylethylamine. DMF=dimethylformamide. DMSO=dimethyl sulfoxide. DPBS=Dulbecco's phosphate buffered saline. EtOAc=ethyl acetate. FBS=fetal bovine serum. FITC=fluorescein isothiocyanate. Fmoc=fluorenylmethoxycarbonyl protecting group. HATU=hexafluorophosphate azabenzotriazole tetramethyl uranium. HLA-A2=human leukocyte antigen-A2. Lawesson's reagent=2,4-bis(4-methoxyphenyl)-1,3,2,4-dithiadiphosphetane-2,4-dithione:

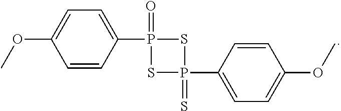

LCMS=liquid chromatography mass spectrometry. MALDI TOF-MS=matrix-assisted laser-desorption ionization time-of-flight mass spectrometry. MHS I=major histocompatibility complex I. Oxyma=ethyl cyanohydroxyiminoacetate. pMHC I=peptide+MHC I complex. S.E.M.=standard error of the mean. TFA=trifluoro acetic acid. THF=tetrahydrofuran. TIPS=triisopropylsilane. TCR=CD8 T cell receptors. TLC=thin layer chromatography.

The term “N-terminus” refers to the end of a polypeptide or protein having a free amine group (—NH₂), an ammonium cation (—NH₃+), or salt thereof. The term “C-terminus” refers to the end of a polypeptide or protein having a free carboxyl group (—COOH), a carboxylate anion (—COO⁻), or salt thereof.

As used herein, the term “C═S-containing residue” refers to an α-amino acid residue in a polypeptide in which the oxygen atom of the backbone carbonyl group is replaced with a sulfur atom. In other words, the present method involves replacing the amide bond between two adjacent residues with a thioamide bond:

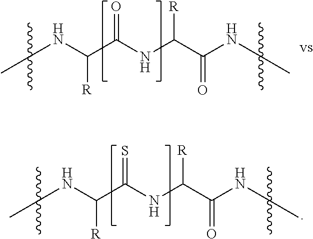

A “corresponding thio-antigen” has the same “R” group (i.e., the same side chain) and the same absolute configuration as the analogous antigen, which is composed exclusively of L-α-amino acid residues.

Sequences:
(SEQ ID NO: 1)
GILGFVFTL
(GIL)
(SEQ ID NO: 2)
GILGFVFTL
(GIL-1)
(SEQ ID NO: 3)
GILGFVFTL
(GIL-2)
(SEQ ID NO: 4)
GILGFVFTL
(GIL-3)
(SEQ ID NO: 5)
GILGFVFTL
(GIL-4)
(SEQ ID NO: 6)
GILGFVFTL
(GIL-5)
(SEQ ID NO: 7)
GILGFVFTL
(GIL-6)
(SEQ ID NO: 8)
GILGFVFTL
(GIL-7)
(SEQ ID NO: 9)
GILGFVFTL
(GIL-8)
(SEQ ID NO: 10)
GILGFVFTL
(GIL-9)
(SEQ ID NO: 11)
GILGFVFTL
(GIL-10)
(SEQ ID NO: 12)
ELAGIGILTV
(ELA)
(SEQ ID NO: 13)
ELAGIGILTV
(ELA-1)
(SEQ ID NO: 14)
ELAGIGILTV
(ELA-2)
(SEQ ID NO: 15)
ELAGIGILTV
(ELA-3)
(SEQ ID NO: 16)
ELAGIGILTV
(ELA-4)
(SEQ ID NO: 17)
ELAGIGILTV
(ELA-5)
(SEQ ID NO: 18)
ELAGIGILTV
(ELA-6)
(SEQ ID NO: 19)
ELAGIGILTV
(ELA-7)
(SEQ ID NO: 20)
ELAGIGILIV
(ELA-8)
(SEQ ID NO: 21)
ELAGIGILTV
(ELA-9)
(SEQ ID NO: 22)
ELAGIGILTV
(ELA-10)
(SEQ ID NO: 23)
AAGIGILTV
(AAG)
(SEQ ID NO: 24)
EAAGIGILTV
(EAA)
(SEQ ID NO: 25)
FLPSDYFPSV

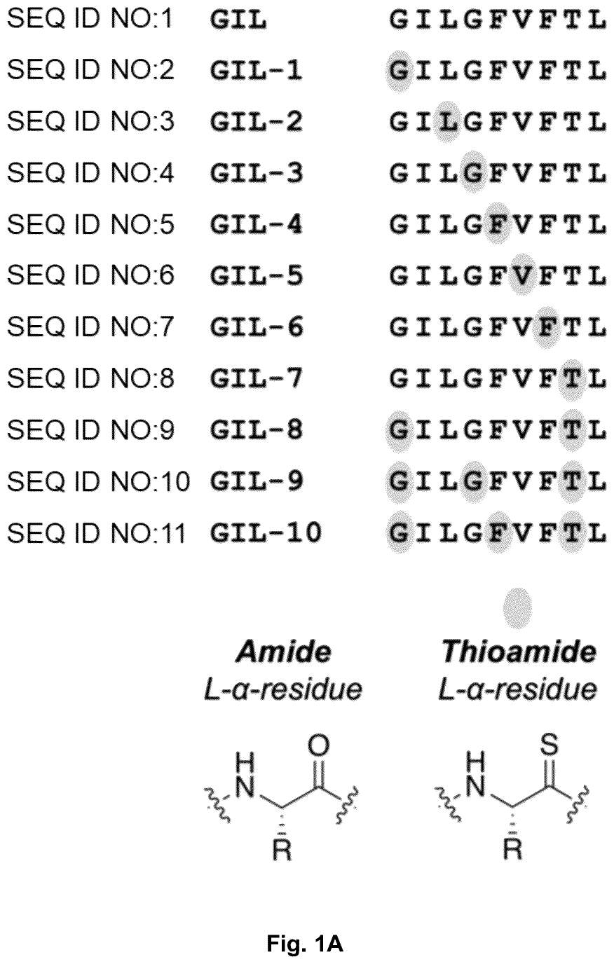

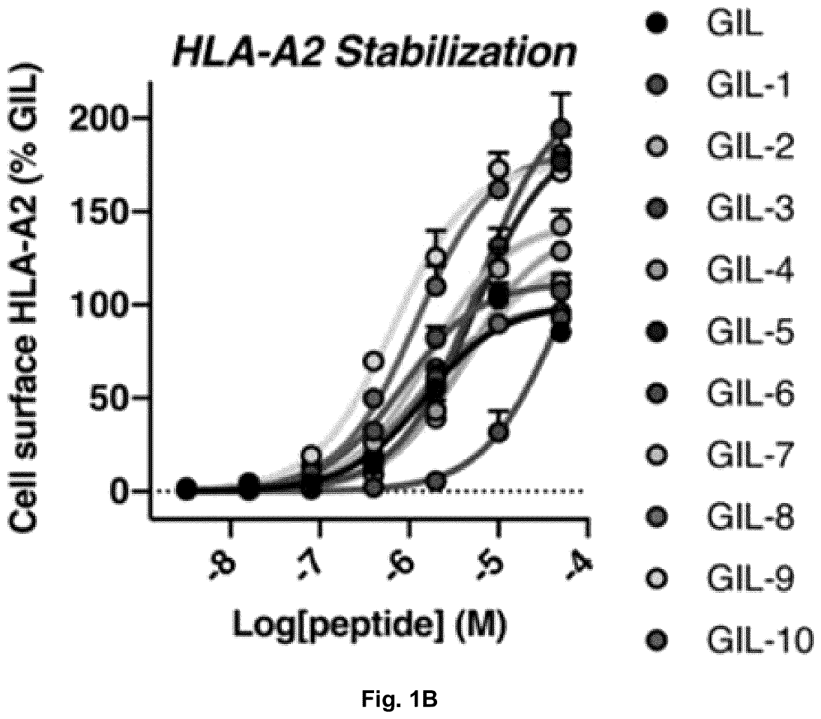

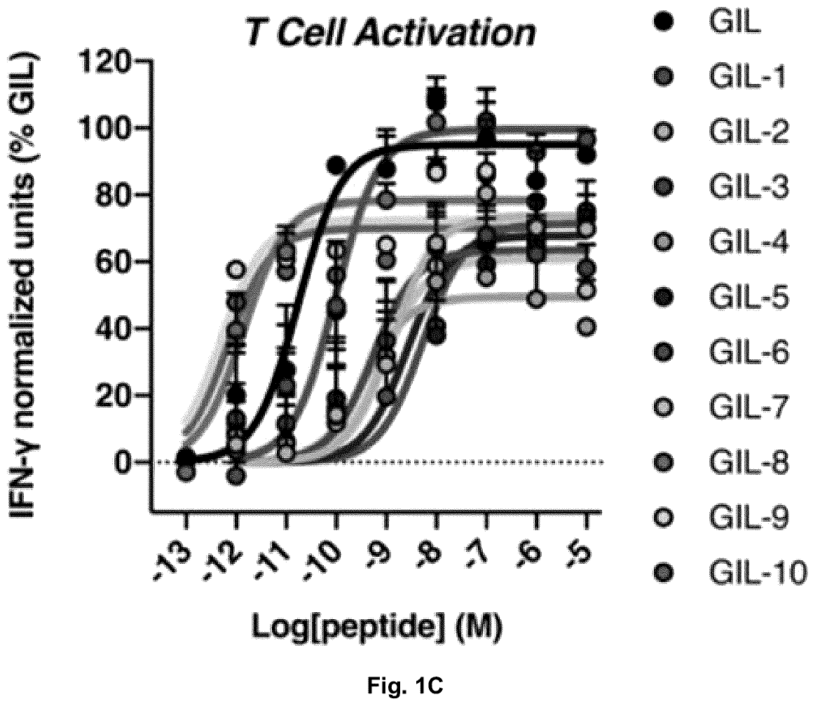

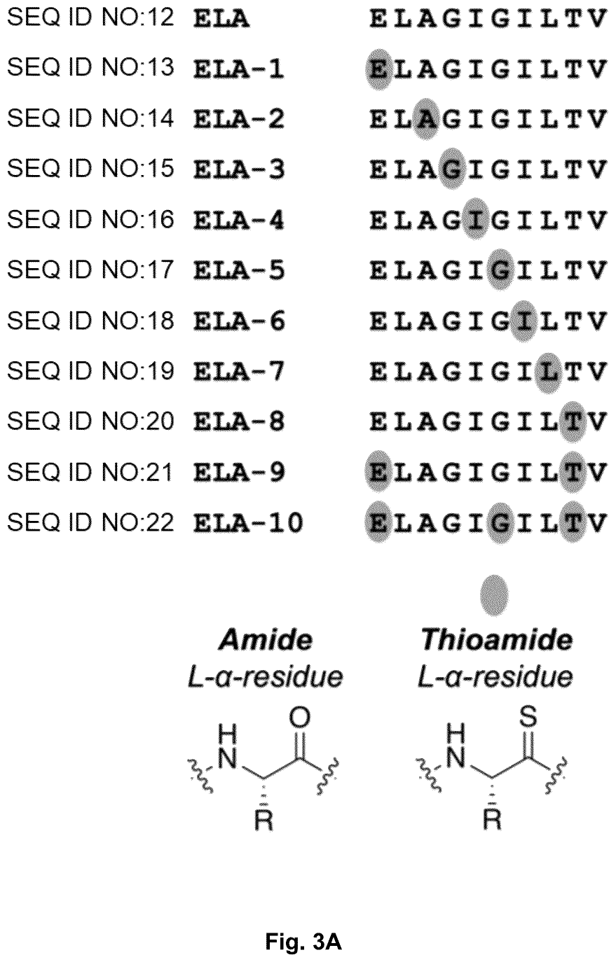

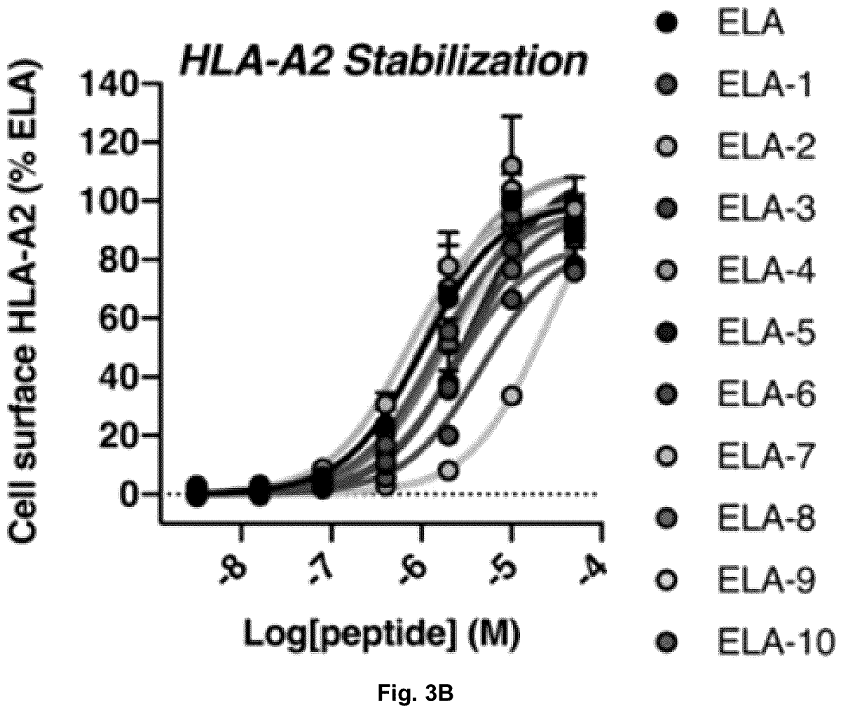

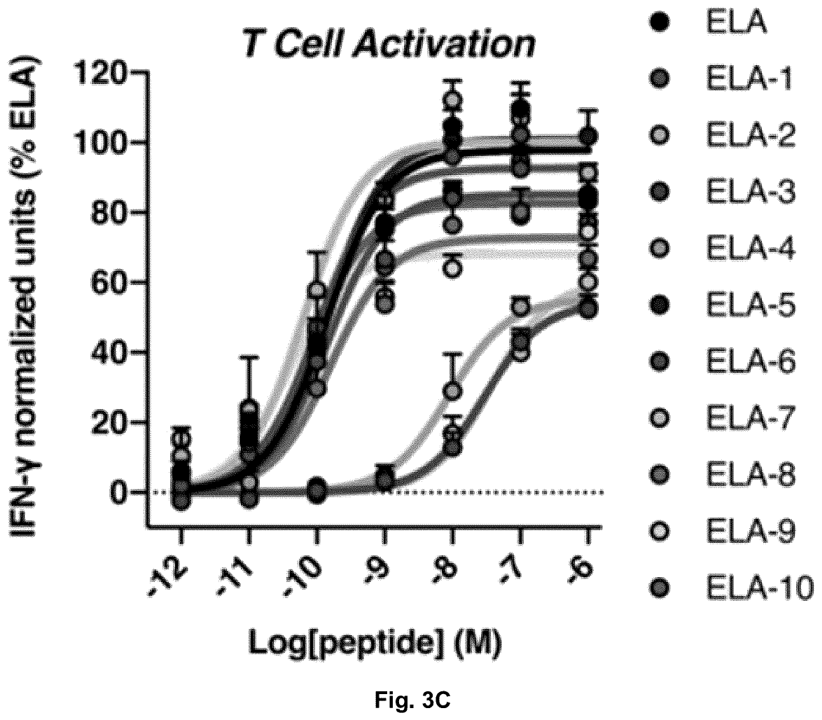

In the above sequences, the bold, underlined residues indicate a residue wherein the backbone C═O group of the native, L-α amino acid residue has been replaced with a backbone C═S group. See, for example, FIGS. 1A and 3A.

### Evaluation of Exemplary Thio-Antigens:

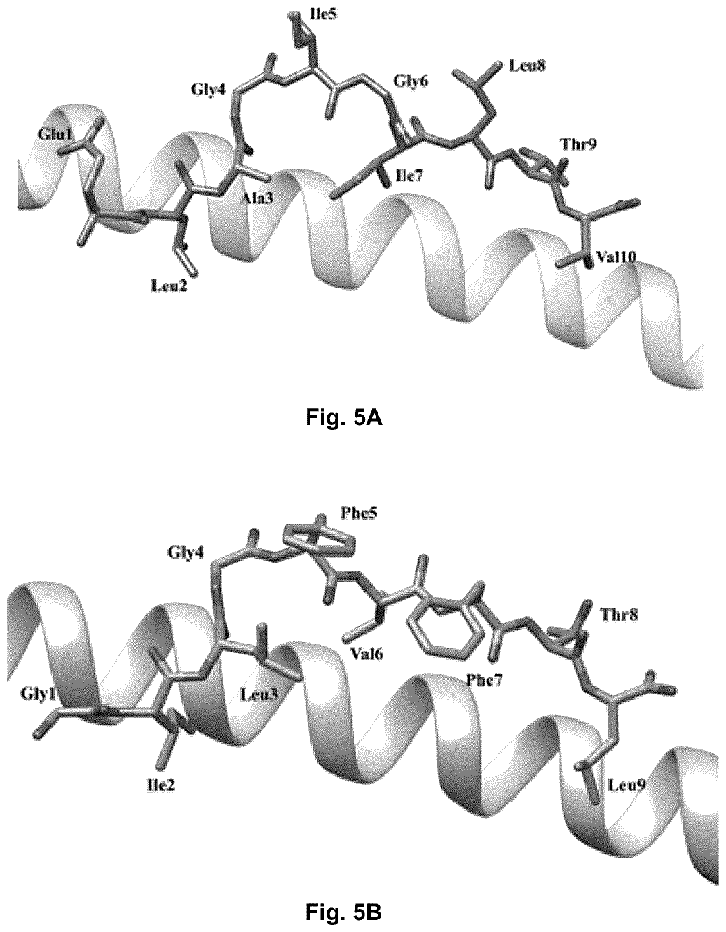

Two well-known HLA-A2-restricted antigens were selected, GILGFVFTL (SEQ ID NO: 1) (“GIL (1-9)” or “GIL”) and ELAGIGILTV (SEQ ID NO:2) (“ELA (1-10)” or “ELA”), for evaluation of the thio-antigen approach. HLA-A2 is found in ˜50% of the human population. Both GIL and ELA have been evaluated in vitro or in clinical trials as potential peptide vaccines. Antigens containing 9 or 10 residues appear to be optimal for HLA-A2 presentation, so one antigen of each length was evaluated. Crystallographic evidence shows that ELA and GIL differ considerably in presentation of the HLA-A2-bound peptide to cognate TCRs (FIGS. 5A-5B). HLAA2-bound ELA adopts a conformation common among MHC I antigens, with a central bulge that protrudes away from the binding groove on HLA-A2 (FIG. 5A). This bulged segment makes contacts with the TCR. In contrast, GIL in complex with HLA-A2 lacks a central bulge, and the central residues of GIL are oriented toward HLA-A2 rather than the TCR (FIG. 5B). Because of these structural differences, it was reasoned that results obtained for thioamides derived from both antigens would collectively test the generality of the thio-antigen design hypothesis. Beyond these two examples of HLA-A2-bound peptide conformations, additional examples of uncommon peptide conformations when bound to the MHC I have been reported. See Hopkins, J. R.; MacLachlan, B. J.; Harper, S.; Sewell, A. K.; Cole, D. K. Unconventional Modes of Peptide-HLA-I Presentation Change the Rules of TCR Engagement. Discovery Immunology 2022, 1 (1), 1-11.

Thiobenzotriazolide derivatives of L-α-amino acids, with Fmoc protection of the backbone amine, were used for incorporation of thioamide units via solid-phase synthesis. Initially, a single thioamide group was introduced at most sites within GIL or ELA. In each case, thioamides at primary anchor residues were not examined, i.e., p2 or p9 for GIL, or p2 or p10 for ELA. Side chains of anchor residues project into pockets at the bottom of the antigen recognition groove of the MHC I. Engagement of these pockets is critical for the stability of the pMHC I complex.

Three assays were used to assess the performance of the thio-antigens. The first measured the ability of thio-antigens to stabilize HLA-A2 on the surface of T2 cells. Peptides that bind with higher affinity to HLA-A2 will generate greater levels of pHLA-A2 complexes on the surface of these cells. The amount of pHLA-A2 on the cell surface can be quantified by flow cytometry using a fluorescently labeled anti-HLA-A2 antibody. The data generated by varying the thio-antigen concentration enabled determination of EC₅₀ (i.e., the thio-antigen concentration required to produce half-maximal response) and % Max (i.e., the maximum amount of HLA-A2 stabilized by a peptide). The natural antigens, GIL and ELA, were used to define 100% response for each comparative dataset. See Ekeruche-Makinde, J., et al. Peptide Length Determines the Outcome of TCR/Peptide-MHCI Engagement. Blood 2013, 121 (7), 1112-1123, and Miles, et al. Real Time Detection of Peptide-MHC Dissociation Reveals That Improvement of Primary MHC-Binding Residues Can Have a Minimal, or No, Effect on Stability. Mol Immunol 2011, 48 (4), 728-732.

To complement the cell-based HLA-A2 stabilization measurements, binding of the thio-antigens was examined using purified HLA-A2 complexes. These experiments measured the ability of each thio-antigen to compete with a radiolabeled peptide known to bind strongly to HLA-A2 (the Phe6→Tyr analogue of HBV core 18-27, FLPSDYFPSV (SEQ ID NO:25), for which KD=2.5 nM). (Miles et al., 2011, supra). These measurements provided IC₅₀ values (i.e., the thio-antigen concentration required to produce half-maximal inhibition of radiolabeled tracer binding).

T cell activation assays were carried out using T2 cells as the antigen-presenting cells and antigen-specific CD8 T cells, which can recognize the pHLA-A2 complex displayed by the T2 cells. The CD8 T cells displayed specificity for either the GIL or ELA antigen; therefore, our assays determined how well thio-antigens derived from GIL or ELA mimic the parent antigen in terms of recognition by a cognate TCR in the context of the pHLA-A2 complex. T cell response was quantified by measuring levels of secreted IFN-γ using ELISA. The data generated by varying the thio-antigen concentration enabled determination of EC₅₀ and % Max values; the maximal responses with GIL or ELA were defined as 100%.

### Thio-Antigen Analogues of GIL

GIL is a highly conserved immunodominant epitope derived from the influenza A virus M1 protein. Unlike most antigens presented by HLA-A2, which form a central bulge that contacts the TCR, GIL is presented with side chains from the central region inaccessible to the TCR; only the p8 Thr side chain of GIL (1-9) located at the C terminus is exposed to solvent (FIG. 5B). This manner of antigen presentation is evident in multiple pMHC I+TCR structures involving the JM22, F50, F6 or F8 TCR, all of which have evolved to recognize HLA-A2-bound GIL largely via the peptide backbone.

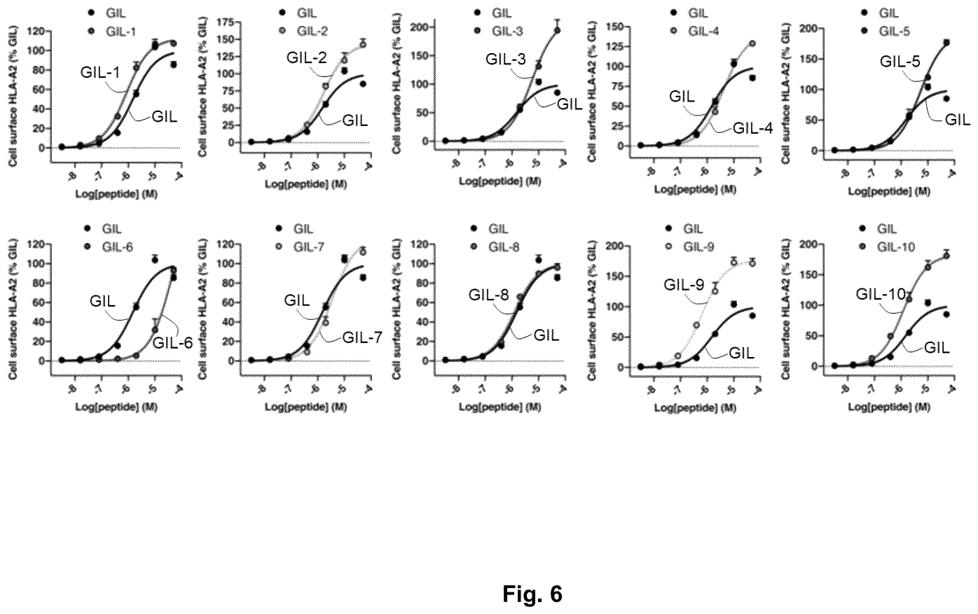

Nearly all the singly-substituted GIL derivatives (GIL-1 to GIL-7; FIG. 1A) were comparable in potency to GIL itself in terms of stabilizing HLA-A2 on the T2 cell surface, as indicated by EC₅₀ values (FIG. 1B; FIG. 6; Table 1). The lone exception to this trend was GIL-6, which contained O→S replacement at p7, a known HLA-A2 secondary anchor position. (Parker, et al. Sequence Motifs Important for Peptide Binding to the Human MHC Class I Molecule, HLA-A2. The Journal of Immunology 1992, 149 (11), 3580-3587.) Each singly-substituted thio-antigen achieved a maximum HLA-A2 level on the cell surface comparable to or higher than that achieved by the native antigen. Those analogues that exceeded the maximum achieved with GIL may form a more stable complex with HLA-A2 relative to the native antigen as a result of C═O to C═S substitution.

The in vitro HLA-A2 affinity assay is considerably more sensitive than the HLA-A2 stabilization assay involving T2 cells. (Gibadullin, R.; Randall, C. J.; Sidney, J.; Sette, A.; Gellman, S. H. Backbone Modifications of HLA-A2-Restricted Antigens Induce Diverse Binding and T Cell Activation Outcomes. J Am Chem Soc 2021, 143 (17), 6470-6481.) The range of IC₅₀ values observed among the seven GIL analogues containing a single thioamide was larger than the range of EC₅₀ values from the T2 assay (Table 1). These two assay formats were consistent in identifying GIL-6 as having substantially lower affinity relative to all other members of this thio-antigen series. The in vitro assay suggested that GIL-1 and GIL-3 bind to HLA-A2 with significantly higher affinity than the native GIL antigen; however, this feature was not evident in the T2 assay (Table 1). These observations raise the possibility that some thioamide locations can increase affinity of a peptide for the MHC I relative to the native antigen.

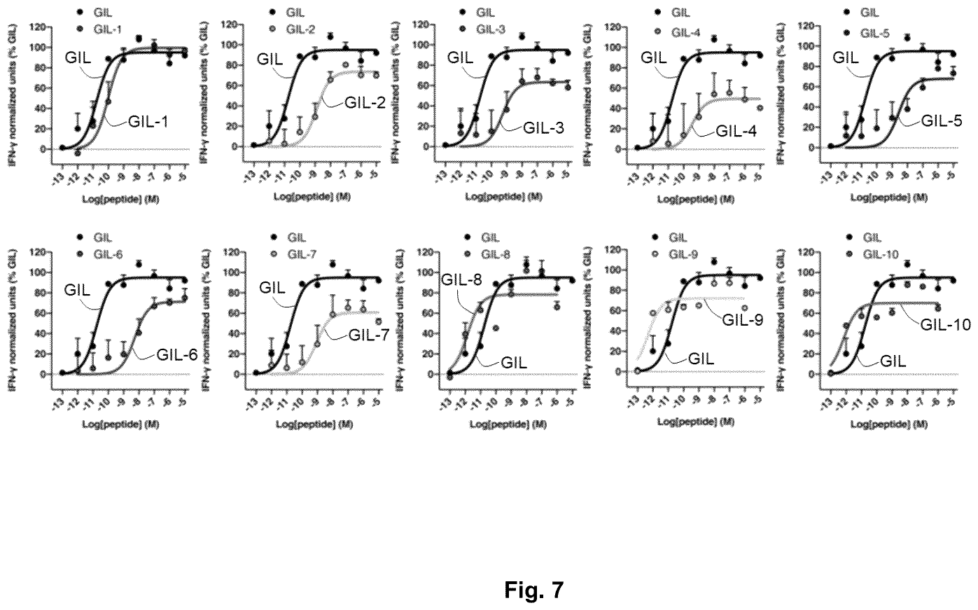

Each of the singly-substituted GIL analogues stimulated IFN-γ secretion from CD8 T cells, but none matched the potency of the native antigen in T cell activation, as manifested by EC₅₀ values (FIG. 1C; FIG. 7; Table 2). The most potent among these analogues was GIL-1, which was the only GIL analogue containing a single thioamide to achieve a maximum in IFN-γ secretion that was comparable to the maximum with the native antigen. GIL-2 to GIL-7 displayed EC₅₀ values 20- to 300-fold higher than that of the native antigen, and they achieved maxima 50% to 74% of the native maximum.

A single thioamide might not offer protection from proteolysis across the entire length of a 9-mer peptide; therefore, three analogues of GIL were prepared that contained multiple thioamides (FIG. 1A). The sites of substitution were selected based on the T cell activation results obtained for the single substitution series (FIG. 1C; FIG. 7; Table 2). GIL-8 contains one thioamide near each end. GIL-9 and GIL-10 share these two terminal substitutions and contain a third substitution near the center. All three were comparable to the native antigen in the HLA-A2 stabilization assay with T2 cells, and all three displayed moderately lower affinity (approximately six-fold higher IC₅₀) in the in vitro HLA-A2 binding assay (FIG. 1B; FIG. 6; Table 1). It was unexpected and unpredictable to observe that GIL-8, GIL-9 and GIL-10 were significantly more potent than the native antigen in the T cell activation assay, although in each case the maximum IFN-γ secretion was modestly lower than for the native antigen (FIG. 1C; FIG. 7; Table 2). The consistently low EC₅₀ values observed for the thio-antigens containing multiple substitutions raise the possibility of favorable cooperativity among the thioamide units in terms of T cell activation.

TABLE 1
HLA-A2 stabilization and in vitro HLA-A2 affinity
responses for GIL and thio-antigens.

| | | | | In vitro Competition | |
|---|---|---|---|---|---|
| | HLA-A2 Stabilization | | | HLA-A2 Affinity | |
| Peptide | EC₅₀ (μM) | EC₅₀ relative | % Max | IC₅₀ (nM) | IC₅₀ relative |
| GIL | 1.5 | 1 | 100 ± 4 | 16 | 1 |
| GIL-1 | 0.84 | 0.6 | 112 ± 3 | <0.25 | <0.016 |
| GIL-2 | 1.7 | 1 | 144 ± 5 | 34 | 2 |
| GIL-3 | 5.6 | 4 | 194* | <0.25 | <0.016 |
| GIL-4 | 4.1 | 3 | 141 ± 4 | 71 | 4 |
| GIL-5 | 5.2 | 4 | 176* | 112 | 7 |
| GIL-6 | 49 | 30 | 93* | 2600 | 160 |
| GIL-7 | 3.5 | 2 | 126 ± 5 | 150 | 9 |
| GIL-8 | 1.2 | 0.8 | 100 ± 2 | 99 | 6 |
| GIL-9 | 0.67 | 0.4 | 177 ± 5 | 88 | 6 |
| GIL-10 | 1.2 | 0.8 | 183 ± 5 | 94 | 6 |

Left: HLA-A2 stabilization EC₅₀ and % Max values are obtained from ≥2 independent experiments. EC₅₀ relative implies the HLA-A2 stabilization potency normalized to GIL: (thioamide analogue/GIL). % Max uncertainties are expressed as S.E.M.
*Value shown indicates % Max at the highest peptide concentration (50 μM); the fitted curve did not reach a saturation point.
Right: In vitro HLA-A2 affinity data are represented by IC₅₀ values obtained from ≥6 independent experiments. IC₅₀ relative indicates the HLA-A2 affinity normalized to GIL by the quotient (thioamide analogue/GIL). GIL-1 and GIL-3 bound HLA- A2 with an affinity below the threshold of the assay, as indicated by approximated values: <0.25 and <0.016 IC₅₀ and IC₅₀ relative, respectively.

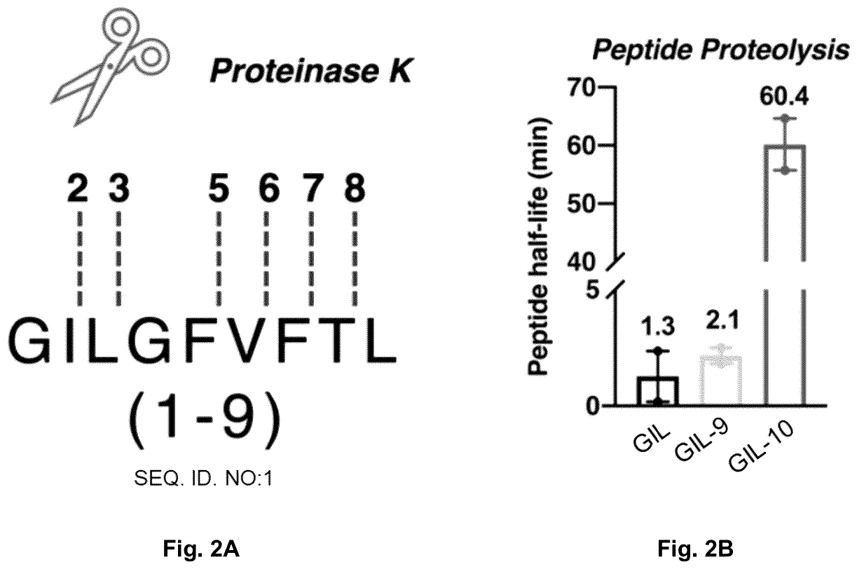

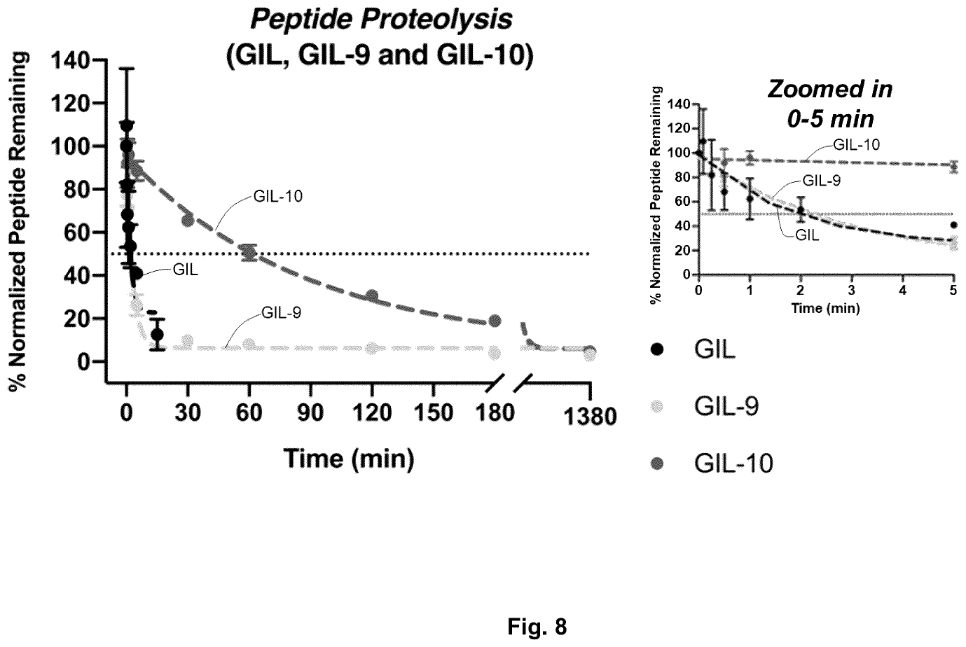

The susceptibility of GIL-9 and GIL-10 to proteolysis was investigated using proteinase K, an aggressive protease with low substrate specificity. The native antigen was used as a point of comparison. The PeptideCutter tool in Expasy predicted six cleavage sites for GIL (after p2, p3, p5, p6, p7 and p8; FIG. 2A). Under our assay conditions, GIL displayed a half-life of 1.3 min (FIG. 2B; FIG. 8). GIL-9, with thioamides at p1, p4 and p8, was not significantly protected from cleavage by proteinase K relative to GIL (half-life of 2.1 min; FIG. 2B, FIG. 8). Shifting the central thioamide by one position, to generate GIL-10, led to a significant increase in half-life (60.4 min; FIG. 2B; FIG. 8), suggesting that locations of thioamide replacements are critical for achieving global proteolytic protection.

TABLE 2
T cell activation responses for GIL and thio-antigens
derived from dose-responses in FIG. 1C.

| | T Cell Activation | | |
|---|---|---|---|
| Peptide | EC₅₀ (pM) | EC₅₀ relative | % Max |
| GIL | 17 | 1 | 95 ± 4 |
| GIL-1 | 97 | 5.7 | 100 ± 5 |
| GIL-2 | 1300 | 76 | 74 ± 5 |
| GIL-3 | 540 | 32 | 63 ± 7 |
| GIL-4 | 370 | 22 | 50 ± 9 |
| GIL-5 | 2600 | 150 | 68 ± 8 |
| GIL-6 | 5300 | 310 | 71 ± 7 |
| GIL-7 | 850 | 50 | 61 ± 6 |
| GIL-8 | 1.4 | 0.082 | 78 ± 6 |
| GIL-9 | 0.49 | 0.029 | 72 ± 4 |
| GIL-10 | 0.69 | 0.041 | 70 ± 4 |

T cell activation EC₅₀ and % Max values are derived from ≥2 independent experiments. EC₅₀ relative implies the T cell activation potency normalized to GIL: (thioamide analogue/GIL). % Max uncertainties are expressed as S.E.M.

### Thio-Antigen Analogues of ELA

ELA is an anchor-modified heteroclitic variant of two natural epitopes derived from the melanoma antigen A protein, AAGIGILTV (SEQ ID NO:23) (AAG) and EAAGIGILTV (SEQ ID NO:24) (EAA). Replacement of Ala with Leu at position 2 causes ELAGIGILTV (SEQ ID NO:12) to bind HLA-A2 with increased affinity relative to either of the natural antigens. Enhanced HLA-A2 binding may explain why ELA is more immunogenic than natural antigen, i.e., why ELA induces far greater frequencies of antigen-specific CD8 T cells both in vitro and in vivo compared to AAG or EAA. CD8 T cells elicited by vaccination with ELA induce lysis of cells that display the natural EAA antigen in pMHC I complexes. HLA-A2-bound ELA adopts a bulged conformation in complex with the MEL5 TCR (FIG. 5A); this TCR was isolated from the CD8 T cell clone generated by stimulating peripheral blood mononuclear cells of a healthy HLA-A2 donor with the ELA peptide. The TCR is centered on the bulged region, residues 4-7 of ELA, and these residues make the majority of peptide-TCR contacts. See Cole, et al. Germ Line-Governed Recognition of a Cancer Epitope by an Immunodominant Human T-Cell Receptor. J Biol Chem 2009, 284 (40), 27281-27289. The architecture of the ELA pMHC I+TCR complex differs from the architecture observed in multiple GIL pMHC I+TCR complexes, as noted above. See, for example, Valkenburg, et al. Molecular Basis for Universal HLA-A*0201-Restricted CD8+ T-Cell Immunity against Influenza Viruses. Proc Natl Acad Sci USA 2016, 113 (16), 4440-4445.

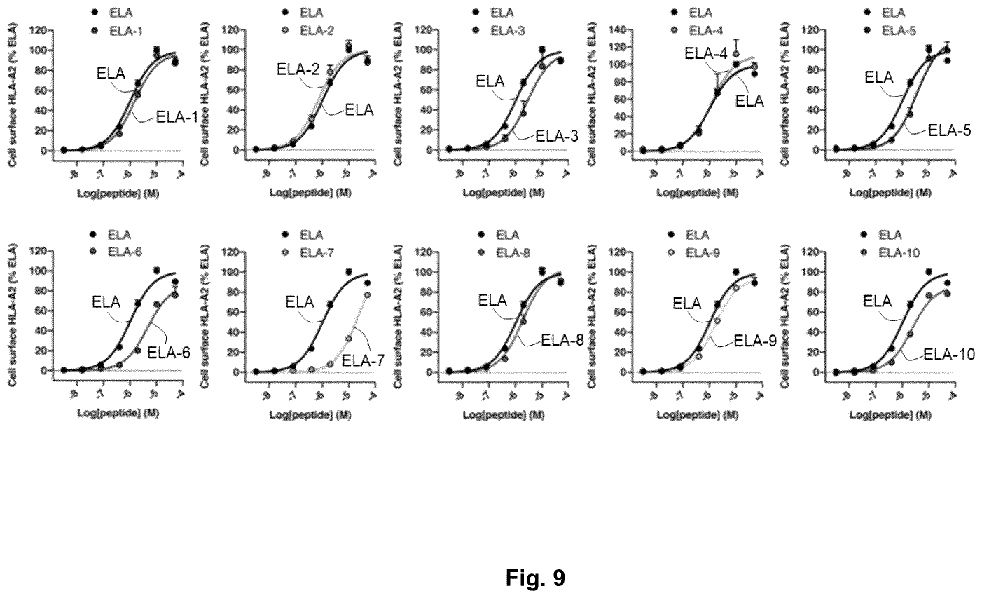

Nearly all the singly-substituted thio-antigens (ELA-1 to ELA-8, FIG. 3A) were comparable in potency to ELA itself in stabilizing HLA-A2 on the surface of T2 cells, as revealed by the EC₅₀ values in Table 3. This trend parallels the present observations for the GIL series (Table 1). The one exception to this trend, ELA-7, resulted from placing the thioamide at the third position from the C-terminus, as was previously observed with GIL-6. Maximum HLA-A2 stabilization was very similar among all singly-substituted thio-antigens except ELA-7 (FIG. 3B; FIG. 9; Table 3).

The in vitro binding assay revealed a slightly larger spread of IC₅₀ values for ELA-1 to ELA-8, ˜40-fold, which contrasts with the ˜20-fold spread among the HLA-A2 stabilization EC₅₀ values. This trend displays the greater sensitivity of the in vitro assay relative to the T2 cell assay that was observed also in the GIL series (Table 1). The two assays that probe the stability of the pHLA-A2 complex are consistent in identifying ELA-7 as the weakest binder among the singly-substituted ELA thio-antigens. ELA-4 appears to bind more tightly than ELA itself in the in vitro assay (Table 3).

TABLE 3
HLA-A2 stabilization and in vitro HLA-A2 affinity
responses for ELA and thio-antigens.

| | | | | In vitro Competition | |
|---|---|---|---|---|---|
| | HLA-A2 Affinity | | | HLA-A2 Stabilization | |
| Peptide | EC₅₀ (μM) | EC₅₀ relative | % Max | IC₅₀ (nM) | IC₅₀ relative |
| ELA | 1.0 | 1 | 100 ± 2 | 42 | 1 |
| ELA-1 | 1.5 | 2 | 97 ± 4 | 200 | 5 |
| ELA-2 | 0.71 | 0.7 | 100 ± 4 | 95 | 2 |
| ELA-3 | 2.9 | 3 | 98 ± 7 | 130 | 3 |
| ELA-4 | 1.2 | 1 | 111 ± 8 | 6.9 | 0.2 |
| ELA-5 | 3.4 | 3 | 111 ± 7 | 100 | 2 |
| ELA-6 | 4.7 | 5 | 87 ± 5 | 580 | 14 |
| ELA-7 | 24 | 20 | 77* | 1500 | 36 |
| ELA-8 | 1.9 | 2 | 104 ± 5 | 33 | 0.8 |
| ELA-9 | 1.7 | 2 | 95 ± 2 | 530 | 13 |
| ELA-10 | 2.3 | 2 | 86 ± 3 | 790 | 19 |

Left: HLA-A2 stabilization EC₅₀ and % Max values are derived from ≥2 independent experiments. EC₅₀ relative implies the HLA-A2 stabilization potency normalized to ELA by the quotient (thioamide analogue/ELA). % Max uncertainties are expressed as S.E.M.
*Value shown describes % Max at the highest peptide concentration (50 μM); the fitted curve did not reach a saturation point.
Right: In vitro HLA-A2 affinity data are represented by mean IC₅₀ values obtained from 6 independent experiments. IC₅₀ relative indicates the HLA-A2 affinity normalized to ELA by the quotient (thioamide analogue/ELA).

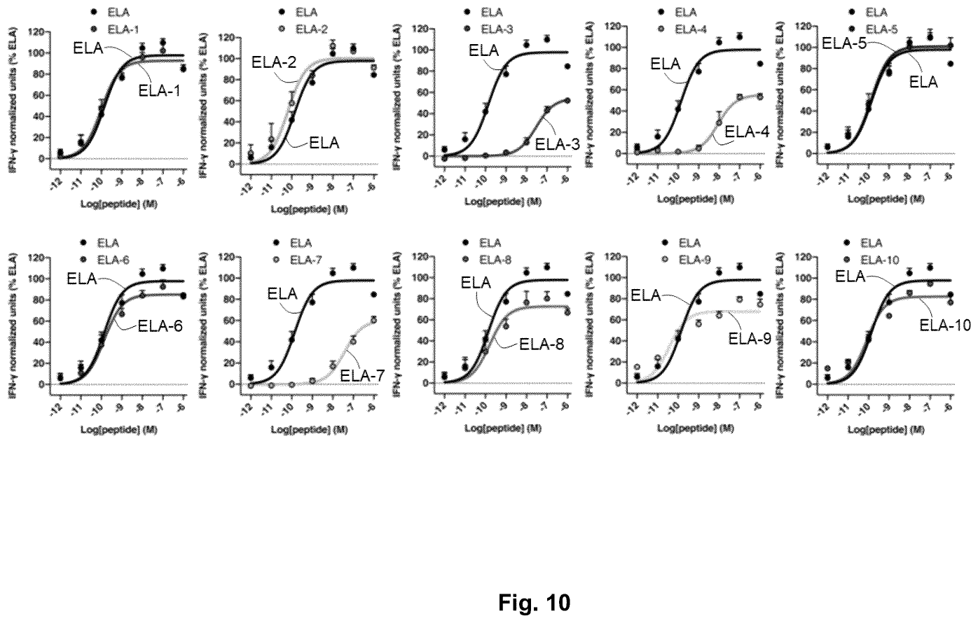

The trend in T cell activation, as indicated by IFN-γ secretion, among singly-substituted ELA thio-antigens differed from the trend observed among GIL thio-antigens. Five among ELA-1 to ELA-8 were comparable to ELA itself in terms of potency, as indicated by EC₅₀ values (FIG. 3C; FIG. 10; Table 4). Among these potent thio-antigens, ELA-1, ELA-2 and ELA-5 matched the maximum achieved by ELA itself; ELA-6 and ELA-8 had moderately lower maxima.

TABLE 4
T cell activation responses for ELA and thio-antigens
derived from dose-responses in FIG. 3C.

| | T Cell Activation | | |
|---|---|---|---|
| Peptide | EC₅₀ (pM) | EC₅₀ relative | % Max |
| ELA | 0.14 | 1 | 98 ± 4 |
| ELA-1 | 0.098 | 0.7 | 93 ± 4 |
| ELA-2 | 0.064 | 0.5 | 100 ± 5 |
| ELA-3 | 29 | 200 | 54 ± 2 |
| ELA-4 | 8.9 | 60 | 55 ± 3 |
| ELA-5 | 0.13 | 0.9 | 101 ± 4 |
| ELA-6 | 0.14 | 1 | 85 ± 3 |
| ELA-7 | 37 | 300 | 60 ± 4 |
| ELA-8 | 0.16 | 1 | 73 ± 4 |
| ELA-9 | 0.037 | 0.3 | 68 ± 3 |
| ELA-10 | 0.081 | 0.6 | 83 ± 4 |

T cell activation EC₅₀ and % Max values are derived from ≥3 independent experiments. EC₅₀ relative implies the T cell activation potency normalized to ELA by the quotient (thioamide analogue/ELA). % Max uncertainties are expressed as S.E.M.

Two ELA analogues containing multiple thioamides were examined: ELA-9, with two C═O to C═S backbone substitutions, one near each terminus, and ELA-10 with a third substitution toward the middle (FIG. 3A). Both were comparable to ELA in the HLA-A2 stabilization assay, but the in vitro binding assay indicated that both of these thio-antigens bound less tightly to HLA-A2 relative to ELA (FIG. 3B; FIG. 9; Table 3). Despite what appeared to be significantly diminished MHC I affinity for ELA-9 and ELA-10 relative to ELA, both of these thio-antigens were comparable to the parent antigen in potency for T cell activation, although the maximum level of activation was somewhat lower for both thio-antigens relative to ELA (FIG. 3C; FIG. 10; Table 4).

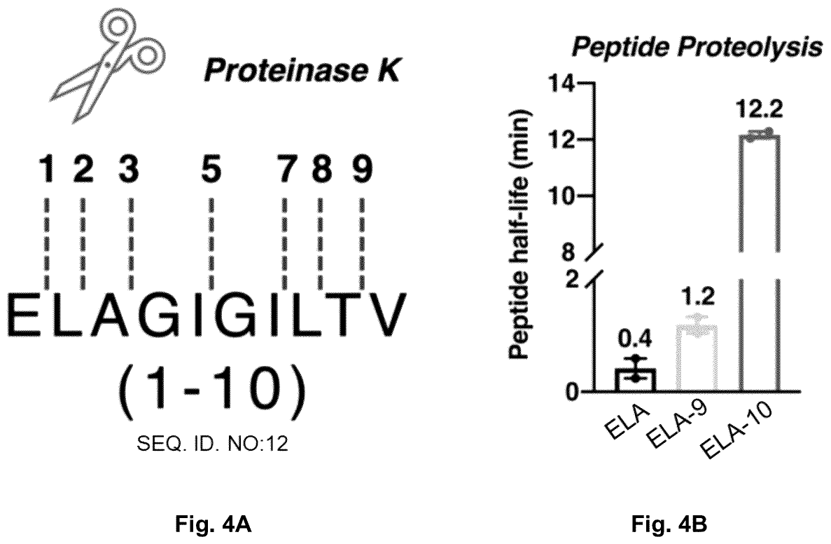

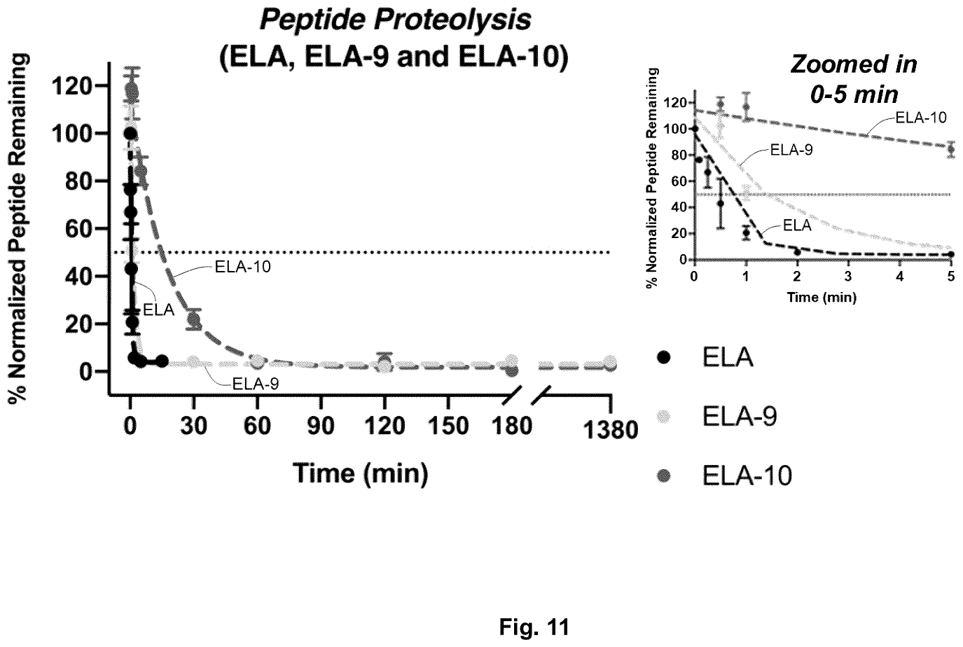

ELA-9 and ELA-10 were evaluated as substrates for proteinase K. The PeptideCutter tool in Expasy predicted seven cleavage sites for ELA (after p1, p2, p3, p5, p7, p8 and p9; FIG. 4A). Under our assay conditions, the half-life of ELA when treated with proteinase K was 0.4 min (FIG. 4B). ELA-9 displayed a modest level of resistance, with a half-life of 1.2 min (FIG. 4B; FIG. 11). Addition of a thioamide near the center of the antigen, to generate ELA-10, increased the half-life to 12.2 min (˜30-fold increase relative to ELA). Thus, as observed in the GIL series, incorporation of three thioamide units dispersed across the antigen can lead to significant protection from proteolysis.

### CONCLUSIONS

Disclosed herein is a new strategy for modifying short peptides that serve as MHC I-displayed antigens with the goal of enhancing immunological function. Specifically, the method yields antigens having a diminished susceptibility to proteolysis while simultaneously retaining recognition of the antigen by HLA-A2 and recognition of the peptide+HLA-A2 complex by cognate T cell receptors. Previous studies have shown that these two recognition events are very sensitive to unnatural modifications of peptide antigens. See, for example, Cheloha, R. W.; Sullivan, J. A.; Wang, T.; Sand, J. M.; Sidney, J.; Sette, A.; Cook, M. E.; Suresh, M.; Gellman, S. H. Consequences of Periodic α-to-β3 Residue Replacement for Immunological Recognition of Peptide Epitopes. ACS Chem Biol 2015, 10 (3), 844-854. Parallel studies with two well-known HLA-A2-restricted antigens, GILGFVFTL (SEQ ID NO:1) and ELAGIGILTV (SEQ ID NO:12), revealed that single C═O to C═S backbone substitutions were tolerated at most positions with only moderate loss in HLA-A2 affinity, as indicated by two complementary assays (Table 1; Table 3). In some cases, however, a single thioamide substitution could substantially enhance or substantially diminish HLA-A2 affinity. A larger range of effects was observed for the single thioamide substitutions in terms of T cell activation, which was monitored via IFNγ secretion (Table 2; Table 4).

Based on the T cell activation results of single amide-to-thioamide substitutions, GIL- or ELA-based thio-antigens were designed that contained two or three substitutions. These thioantigens displayed modestly diminished HLA-A2 affinity relative to the native antigen, but they were quite potent in activating cognate T cells (FIGS. 1A-1C; FIGS. 3A-3C; Tables 1-4). Both of the GIL thio-antigens with three substitutions (GIL-9 and GIL-10) were significantly more potent than the native antigen in terms of stimulating IFN-γ secretion from T cells (lower EC₅₀ values), and the ELA thio-antigen with three substitutions (ELA-10) was slightly more potent in stimulating IFN-γ secretion relative to the native antigen (Tables 2 and 4). In both systems, the maximum level of IFN-γ secretion was moderately lower for the thio-antigens relative to the parent antigen. The T cell activation potency of the triply-substituted analogues was not predictable based on the behavior of the corresponding singly-substituted analogues. Thus, there appears to be a synergistic effect from multiple amide-to-thioamide replacements.

Substantial protection from degradation by proteinase K was observed for one of the triply-substituted GIL thio-antigens and for the triply-substituted ELA thio-antigen. The combination of resistance to proteolysis and potent T cell activation observed for two different HLA-A2 antigens indicates that the thio-antigen design strategy is broadly applicable for generating CD8 T cell-directed vaccines.

### Materials and Methods:

TABLE 5
Materials.

| | | CATALOG #/ |
|---|---|---|
| REAGENT/RESOURCE | SOURCE | IDENTIFIER |
| Peptide Synthesis | | |
| α-N—Fmoc-L-amino acids | Chem Impex (Wood Dale, Illinois, USA); | Various |
| | AAPPTec, LLC (Louisville, Kentucky, | |
| | USA) | |
| Acetonitrile HPLC-grade | Millipore Sigma (Burlington, | 34851 |
| | Massachusetts, USA) | |
| Ethyl (hydroxyimino) cyanoacetate | Chem Impex | 26426 |
| (Oxyma) | | |
| N₃N′-Diisopropylcarbodiimide | Chem Impex | 00110 |
| (DIC) | | |
| Piperidine ReagentPlus ® | Millipore Sigma | 104094 |
| α-Cyano-4-hydroxycinnamic acid | Millipore Sigma | 70990 |
| (CHCA) | | |
| SPPS reaction vessel syringes/caps | Torviq (Tucson, Arizona, USA) | SF-1000/PC-SF |
| N₃N Dimethylformamide (DMF) | Millipore Sigma | 494488 |
| biotech-grade | | |
| N₃N Dimethylformamide (DMF) | Millipore Sigma | 319937 |
| ACS-grade | | |
| Trifluoroacetic acid (TFA) | Millipore Sigma | T6508 |
| ReagentPlus ® | | |
| 1,2 ethanedithiol | Millipore Sigma | 02390 |
| Thioanisole ReagentPlus ® | Millipore Sigma | T28002 |
| Triisopropylsilane (TIPS) | Millipore Sigma | 233781 |
| Fmoc-Leu-Wang resin | AAPPTec | RWL101 |
| Fmoc-Val-Wang resin | AAPPTec | RWV101 |
| Cell Assays | | |
| T2 cells (174 × CEM.T2) | ATCC (Gaithersburg, Maryland, USA) | CRL-1992 |
| GILGFVFTL (SEQ ID NO: 1) | Charles River Laboratories Cell Solutions | ASTC-1039 |
| (Influenza/M1) antigen-specific | (Wilmington, Massachusetts, USA) | |
| CD8 T cells | | |
| ELAGIGILTV (SEQ ID NO: 12) | Charles River Laboratories Cell Solutions | ASTC-1072 |
| (Melanoma/MART-1) | | |
| antigen-specific CD8 T cells | | |
| Human IFN-gamma DuoSet | R&D Systems (Minneapolis, Minnesota, | DY285B-05 |
| ELISA | USA) | |
| DuoSet ELISA Ancillary Reagent | R&D Systems | DY008B |
| Kit 2 | | |
| Dulbecco's Phosphate Buffered | Millipore Sigma | D8537 |
| Saline (DPBS) | | |
| HyClone ™ Penicillin-Streptomycin | GE Healthcare Life Sciences (Wauwatosa, | SV30010 |
| Solution (100×) | Wisconsin, USA) | |
| Fetal Bovine Serum (FBS) | Gibco/Corning | 10082147/45000-734 |
| β2-microglobulin | Lee BioSolutions | 126-11-1 |
| Bovine serum albumin stain buffer | BD Biosciences | 554657 |
| RPMI 1640 medium | ATCC | 30-2001 |
| FITC-labeled anti-human HLA-A2 | Fisher Scientific | BDB551285 |
| antibody (clone BB7.2) | | |
| X-Vivo 15 serum-free | Lonza Bioscience | 04-418Q |
| hematopoietic cell medium | | |
| Gibco CTS AIM-V medium | Fisher Scientific | A3830801 |
| without phenol red and antibiotics | | |
| Non-treated clear 96-well plates | CellTreat Scientific Products | 229590 |
| (round bottom) | | |
| Nunc non-treated T75 suspension | Fisher Scientific | 1256685 |
| flasks | | |
| Costar 50 mL Sterile Reagent | DOT Scientific | 4870 |
| Reservoirs | | |
| Proteolysis | | |
| Proteinase K Lyophilized | Millipore Sigma | 70663-4 |
| Acetaminophen BioXtra | Millipore Sigma | A7085 |
| Softwares and Illustrations | | |
| GraphPad Prism 8 | GraphPad Software, LLC (Boston, | N/A |
| | Massachusetts, USA) | |
| Microsoft PowerPoint 16.73 | Microsoft (Redmond, Washington, USA) | N/A |
| ChemDraw 22.0.0 | PerkinElmer | N/A |
| FloJo 10.7.1 | FloJo, LLC | N/A |
| UCSF Chimera 1.16 | Resource for Biocomputing, Visualization, | N/A |
| | and Informatics (RBVI), UCSF | |
| Synthesis of Thiobenzotriazolide | | |
| Derivatives of L-α-amino Acids | | |
| 4-nitro-o-phenylenediamine | Fisher Scientific | AC148841000 |
| Lawesson's Reagent | Fisher Scientific | AC210890250 |
| O-(7-Azabenzotriazol-1-yl)- | Chem Impex | 12881 |
| N,N,N′,N′-tetramethyluronium | | |
| hexafluorophosphate (HATU) | | |
| DIEA | Fisher Scientific | AAA11801AE |
| Sodium Nitrite | Fisher Scientific | 749716 |

TABLE 6
Source and donor information for antigen-
specific CD8 T cells obtained from
Charles River Laboratories Cell Solutions.

| | | Donor |
|---|---|---|
| HLA-A2 Antigen | Catalog # | ID |
| GILGFVFTL | ASTC-1039 | 401 |
| (SEQ ID NO: 1) | Charles River Laboratories | |
| | Cell Solutions | |
| | (Wilmington, | |
| | Massachusetts, USA) | |
| ELAGIGILTV | ASTC-1072 | 358 |
| (SEQ ID NO: 12) | Charles River Laboratories | |
| | Cell Solutions | |

### Synthesis of Thiobenzotriazolide Derivatives of L-α-Amino Acids

Thiobenzotriazolide derivatives of L-α-amino acids in Fmoc-protected form were synthesized in three steps according to previously described methods. (Khatri, B.; Bhat, P.; Chatterjee, J. Convenient Synthesis of Thioamidated Peptides and Proteins. Journal of Peptide Science 2020, 26 (4-5), e3248. Mukherjee, S.; Verma, H.; Chatterjee, J. Efficient Site-Specific Incorporation of Thioamides into Peptides on a Solid Support. Org Lett 2015, 17 (12), 3150-3153.)

Step 1: Fmoc-AA-OH (1 equiv.), where AA is an L-α-amino acid with appropriate side chain protection, was dissolved in DMF (0.1 M). While this solution was stirring, HATU (1.5 equiv.) and DIEA (3 equiv.) were added sequentially. Afterwards, 4-nitro-o-phenylenediamine (1.2 equiv.) was added, and the reaction was allowed to proceed for 6 h. The product was precipitated by addition of cold H₂O, filtered, and washed with cold H₂O. The precipitate was then dissolved in EtOAc, and this solution was washed with 1 M HCl, saturated aqueous NaHCO₃, and saturated aqueous NaCl. The organic layer was then dried over Na₂SO₄ and concentrated in vacuo to yield an orange/yellow crude solid. Note: it was discovered that when MgSO₄ was used as a drying agent instead of Na₂SO₄, the yield of the product was substantially lower.

Step 2: The crude product from step 1 was dissolved in dry THF (0.1 M). Lawesson's reagent (1.5 equiv.) was added, and the reaction was allowed to proceed until completion as indicated by TLC (6-48 h). The reaction mixture was reduced in vacuo, and the residue was redissolved in EtOAc. This solution was washed with saturated aqueous NaHCO₃ and then H₂O. The solution was dried over Na₂SO₄ and concentrated. The residue was purified by column chromatography (eluted with EtOAc/pentanes) to yield an orange foam.

Step 3: The product from step 2 was dissolved in glacial AcOH (95%), and the solution was cooled to 0° C. 1.5 equiv. of sodium nitrite was then added in portions, and the mixture was allowed to stir until the reaction had gone to completion as indicated by TLC (0.5-3 h). The reaction mixture was then diluted with cold H₂O and centrifuged at 4000 rpm for 10 min. The supernatant was decanted, and the remaining solid was dissolved in DCM. This solution was washed with saturated aqueous NaHCO₃, and dried over Na₂SO₄. The DCM solution containing the thiobenzotriazolide derivative of the L-α-amino acid was then used directly for solid phase peptide synthesis.

### Peptide Synthesis

Thioamide analogues (“thio-antigens”) of GILGFVFTL (SEQ ID NO:1) or ELAGIGILTV (SEQ ID NO:2) were synthesized at 50 μmol scale using a preloaded Fmoc-Leu-Wang or Fmoc-Val-Wang resin, respectively. (The resins were purchased commercially from AAPPTec, LLC, Louisville, Kentucky, USA.) The synthesis of thio-antigens required both an automated microwave-assisted coupling/deprotection cycles on a CEM Liberty Blue instrument and manual microwave-assisted coupling/deprotection steps on a CEM MARS 6 instrument (CEM Corporation, Charlotte, North Carolina, USA). For couplings on the Liberty Blue instrument, 1.25 mL of 0.2 M Fmoc-protected amino acid was mixed with 0.5 mL of 0.5 M DIC and 0.5 mL of 1 M Oxyma, all prepared in biotech grade DMF, to provide the activated amino acid solution. Double couplings were performed at 75° C. for 4 min for each amino acid. Deprotections were performed with 20% (v/v) piperidine and 0.1 M Oxyma in ACS-grade DMF at 90° C. for 2 min.

Fmoc-protected thiobenzotriazolide derivatives of L-α-amino acids were coupled manually to the growing peptide chain. Two equivalents of thiobenzotriazolide in DCM was combined with the resin in a syringe with a frit, and the syringe was then placed on a rocker for 45 min at room temperature with protection from light. The coupling solution was then drained, and the coupling step using two equivalents of thiobenzotriazolide was repeated one more time. After coupling the first thioamide residue on the resin, each subsequent Fmoc deprotection was performed twice at room temperature for 30 sec each time using a solution of 10% piperidine in ACS-grade DMF. This change in deprotection protocol was implemented to avoid epimerization at the site of thioamide substitution. All subsequent couplings with Fmoc-protected L-α-amino acids were performed manually at 70° C. for 8 min using a combination of 1.25 mL of 0.2 M Fmoc-protected amino acid residue, 0.5 mL of 1 M DIC and 0.5 mL of 1 M Oxyma.

Cleavage of peptides from the resin was achieved with 3 mL (enough to cover the resin) of 46.4% DCM (v/v), 46.4% TFA (v/v), 2.4% thioanisole (v/v), 2.4% 1,2-ethanedithiol (v/v), and 2.4% TIPS (v/v) at room temperature for 2 h with gentle stirring. The cleavage solution was then filtered, and the filtrate was placed under a gentle stream of N2 to decrease the volume by ˜75%. The crude peptide was precipitated by addition of 35 mL of cold diethyl ether. This material was pelleted by centrifugation at 3500 rpm for 5 min. The supernatant (diethyl ether and cleavage solution) was poured off, and the crude peptide pellet was dried under an N₂ stream.

Preparative reverse-phase HPLC was performed on a Waters HPLC system fitted with a Waters or Supelco C18 column eluted with a 5-95% acetonitrile+0.1% TFA (v/v) gradient in Nanopure water+0.1% TFA (v/v). Purity and characterization for all but one peptide (natural ELA antigen) was assessed by Liquid Chromatography Mass Spectrometry (LCMS) using a Waters Acquity Arc System Analytical HPLC outfitted with an XBridge C18 column (2.1×50 mm). The purity of the ELA peptide was assessed using a Waters Acquity H-Class UPLC outfitted with an Acquity UPLC Peptide BEH C18 column (1.7 μm, 2.1×100 mm). For characterization of ELA peptide, MALDI TOF-MS data was acquired on a Bruker Microflex™ MALDI-TOF mass spectrometer. Purity and characterization data for the native GIL antigen were reported previously. (Gibadullin et al (2021), supra.) The concentration of each thio-antigen was determined by UV-Vis absorbance at 255 nm (ThermoFisher NanoDrop™ 2000 spectrophotometer) using the extinction coefficient reported for a thioamide unit (12,400 M⁻¹ cm⁻¹). (Helbing, J.; Bregy, H.; Bredenbeck, J.; Pfister, R.; Hamm, P.; Huber, R.; Wachtveitl, J.; De Vico, L.; Olivucci, M. A Fast Photoswitch for Minimally Perturbed Peptides: Investigation of the Trans→Cis Photoisomerization of N-Methylthioacetamide. J Am Chem Soc 2004, 126 (28), 8823-8834.)

### Cell Culture

T2 cells were maintained in non-treated T75 suspension culture flasks in RPMI 1640 medium supplemented with 10% FBS and 1% penicillin/streptomycin at 37° C. under 5% CO₂. Cells were passaged at 1/3 dilution every 4-5 days.

### HLA-A2 Stabilization

The assay was performed exactly as previously described. T2 cells grown in T75 flasks were centrifuged at 500 rpm. After the medium was aspirated, cells were washed once in FBS-free RPMI 1640 medium. The T2 cells were plated (100,000 cells/well; 80 μL) using serum-free AIMV medium in black, non-treated 96-well plates with a flat bottom. 10 μL of 30 μg/mL B2-microglobulin dissolved in DPBS was pipetted into each well. Afterwards, 10 μL solutions of various peptide doses prepared in DPBS were added to the cells. The T2 cells were incubated overnight (18 h) at 37° C. in 5% CO₂. The next day, the T2 cells were washed once using bovine serum albumin stain buffer to remove any unbound peptide and B2-microglobulin. The cells were then stained with 5 μg/mL of FITC-labelled anti-human HLA-A2 antibody (BB7.2 clone) at 4° C. for 30 min. Any unbound FITC-labelled antibody was removed by washing the T2 cells with bovine serum albumin stain buffer. Finally, the T2 cells were resuspended in the same stain buffer before data acquisition using a ThermoFisher Attune flow cytometer. Reported EC₅₀ and % Max values were a result of normalizing, averaging, and fitting median fluorescence data for live T2 cells to three-parameter sigmoidal curves in GraphPad Prism 8 (GraphPad Software, Boston, Massachusetts). The bottom of each curve was constrained to 0%. For normalization of individual experiments, 0% was defined as the average median fluorescence in wells containing no peptide; 100% was defined as the calculated top of the native antigen's (GILGFVFTL (SEQ ID NO:1) or ELAGIGILTV (SEQ ID NO:2)) fluorescence curve. Examples of flow cytometry plots used for this type of analysis, involving peptides with affinity for HLA-A2, can be found in the literature. (See the Supporting Information in Gibadullin et al. (2021), supra.)

### In Vitro HLA-A2 Affinity

The assay to quantitatively measure the in vitro affinity (via competition) of a peptide for HLAA2 was performed as described in detail previously. (Gibadullin et al (2021), supra, and Sidney, J.; Southwood, S.; Moore, C.; Oseroff, C.; Pinilla, C.; Grey, H. M.; Sette, A. Measurement of MHC/Peptide Interactions by Gel Filtration or Monoclonal Antibody Capture. Curr Protoc Immunol 2013, 100 (1), 18.3.1-18.3.36.) Purified HLA-A2 molecules were co-incubated at room temperature with HLA-A2-binding radiolabeled peptide (F6→Y analogue of HBV core 18-27; sequence FLPSDYFPSV (SEQ ID NO:25); KD=2.5 nM) and different doses (covering 100,000-fold dose range) of a competitor peptide. Following a 48 h incubation, HLA-A2 binding of the radiolabeled peptide was determined by capturing HLA-A2+peptide complexes on W6/32 antibody-coated Lumitrac 600 plates and measuring bound cpm using a TopCount microscintillation counter. The binding data were analyzed and IC₅₀ values (i.e., the concentration of a competitor peptide that yields 50% inhibition of the binding of the radiolabeled peptide) were determined as previously described.

### T Cell Activation

The assay to measure the ability of T2-loaded peptides to activate antigen-specific CD8 T cells was performed as described previously. (Gibadullin et al (2021), supra.) T2 cells suspended in growth medium (RPMI 1640 supplemented with 10% FBS and 1% penicillin/streptomycin) were centrifuged at 500 rpm before the medium was aspirated. The cells were then washed once in serum-free RPMI 1640 medium and resuspended in DPBS, with cell concentration adjusted to 2×106 cells/mL. Mitomycin C was then added to the T2 cells at a 50 μg/mL final concentration. The cells were incubated for 1 h at 37° C. in 5% CO₂. Afterwards, the T2 cells were washed three times with DPBS, with each wash containing at least 10 mL of DPBS. The inactivated T2 cells were suspended in serum-free X-Vivo 15 medium and plated at 10,000 or 20,000 cells/well (80 μL volume) into clear, non-treated 96-well plates with a round bottom. Different doses of peptides as 20 μL solutions in DPBS were then added to the T2 cells, which were allowed to incubate for 30 min at 37° C. in 5% CO₂. During this time, antigen-specific CD8 T cells were thawed out using X-Vivo 15 medium supplemented with 10% FBS following the manufacturer's directions. After 10 min centrifugation at 1000 rpm, the CD8 T cells were resuspended in serum-free X-Vivo 15 medium, and 10,000 or 20,000 of these T cells (1:1 ratio of T2 cells to CD8 T cells) were added into each well. After overnight incubation (18 h) at 37° C. in 5% CO₂, supernatant from each well was collected. IFN-γ secretion was measured using Human IFN-gamma DuoSet ELISA according to the manufacturer's instructions. Optical density (OD) values were measured at 450 nm using a BioTek Synergy 2 microplate reader. Reported EC₅₀ and % Max values were a result of normalizing, averaging, and fitting OD data to three-parameter sigmoidal curves in GraphPad Prism 8. The bottom of each curve was constrained to 0%. For normalization of individual experiments, 0% was defined as the average OD in wells containing no peptide. 100% was defined as the calculated top of the native antigen's (SEQ ID NO: 1 or SEQ ID NO:2) OD curve.

### Proteolysis

The protocol was adapted from Checco et al. with slight modifications. (Checco, J. W.; Eddinger, G. A.; Rettko, N. J.; Chartier, A. R.; Gellman, S. H. Tumor Necrosis Factor-α Trimer Disassembly and Inactivation via Peptide-Small Molecule Synergy. ACS Chem Biol 2020, 15 (8), 2116-2124.) Stock solutions of 25 μg/mL proteinase K were freshly prepared in DPBS, pH 7.4. 20 μL of 1 mM (20% DMSO/80% DPBS) GIL or ELA antigen peptide or a thioamide analogue was diluted with 60 μL of DPBS, after which 20 μL of proteinase K solution (25 μg/mL) was added. Thus, the final peptide proteolysis solution contained 200 μM of peptide (4% v/v DMSO) and 5 μg/mL of proteinase K. At each time point, a 10 μL aliquot of the proteolysis reaction was removed and mixed with 20 μL of the quenching solution consisting of 49.5% H₂O/49.5% acetonitrile/1% TFA with 40 μg/mL of acetaminophen included as an internal standard. A “0 min” control for each peptide was prepared by mixing 3 μL of 1 mM (20% DMSO/80% DPBS) stock of GIL or ELA or a thioamide analogue with 12 μL of DPBS. A 10 μL aliquot of each quenched reaction solution was injected onto a Waters Acquity H-Class UPLC equipped with an Acquity BEH C18 column. 0-90% B (A: Nanopure water+0.1% TFA; B: HPLC-grade acetonitrile+0.1% TFA) gradient over 10 min with a 0.3 mL/min flow rate was used for elution; the gradient began after the first minute of elution, and during this first minute, the eluent was 100% A. The relative amount of peptide was quantified by integration of the peptide peak at 220 nm and normalization to the acetaminophen internal standard. For each sample, the % peptide remaining was calculated by dividing the normalized peak area by the normalized peak area from the “0 min” control. All proteolysis reactions were run in duplicate. Half-lives were determined by fitting normalized data to a one-phase decay model in GraphPad Prism 8.

What is claimed is:

1. A method to make polypeptide antigens with increased resistance to enzymatic degradation, the method comprising fabricating a polypeptide analogue of a native polypeptide antigen in which at least one backbone C═O group in the native polypeptide antigen residues is replaced with a backbone C═S group, wherein the polypeptide analogue has an N-terminus and a C-terminus.

2. The method of claim 1, wherein at least two backbone C═O groups in the native polypeptide antigen residues are replaced with backbone C═S groups.

3. The method of claim 1, wherein one of the backbone C═S groups is located at the N-terminus of the polypeptide analogue.

4. The method of claim 1, wherein none of the backbone C═S groups are located at the C-terminus of the polypeptide analogue.

5. The method of claim 1, wherein at least two or at least three backbone C═O groups in the native polypeptide antigen residues are replaced with backbone C═S groups.

6. The method of claim 1, wherein no more than and no less than three backbone C═O groups in the native polypeptide antigen residues are replaced with backbone C═S groups.

7. The method of claim 1, wherein the native polypeptide antigen and the polypeptide analogue consist essentially of from 8 to 100 residues.

8. The method of claim 1, wherein the native polypeptide antigen and the polypeptide analogue consist essentially of from 8 to 50 residues.

9. The method of claim 1, wherein the native polypeptide antigen and the polypeptide analogue consist essentially of from 8 to 25 residues.

10. The method of claim 1, wherein the native polypeptide antigen and the polypeptide analogue consist essentially of from 8 to 15 residues.

11. The method of claim 1, wherein at least three backbone C═O groups in the native polypeptide antigen residues are replaced with backbone C═S groups and none of the backbone C═S groups are located at the C-terminus of the polypeptide analogue.

12. The method of claim 11, wherein none of the backbone C═S groups are contiguous to one another.

13. The method of claim 11, wherein at least one of the backbone C═S groups is located at the N-terminus of the polypeptide analogue.

14. A non-natural polypeptide antigen made by the method recited in claim 1.

15. A non-natural polypeptide antigen with increased resistance to enzymatic degradation, the polypeptide antigen comprising from 8 to 100 residues and having an N-terminus and a C-terminus, wherein at least one residue of the polypeptide antigen has a backbone C═O group replaced with a backbone C═S group, and wherein the residue at the C-terminus has a backbone C═O group.

16. The non-natural polypeptide antigen of claim 15, comprising from 8 to 50 residues.

17. The non-natural polypeptide antigen of claim 15, comprising from 8 to 25 residues.

18. The non-natural polypeptide antigen of claim 15, comprising from 8 to 15 residues.

19. The non-natural polypeptide antigen of claim 15, wherein the residue at the N-terminus has a backbone C═S group.

20. The non-natural polypeptide antigen of claim 19, wherein all other residues are α-amino acid residues.

21. The non-natural polypeptide antigen of claim 20, wherein the α-amino acid residues are L-α-amino acid residues.

22. The non-natural polypeptide antigen of claim 15, wherein the residues having backbone C═S groups are derived from L-α-amino acid residues.

23. The non-natural polypeptide antigen of claim 15, wherein at least two residues are residues in which a backbone C═O group is replaced with a backbone C═S group.

24. The non-natural polypeptide antigen of claim 15, wherein at least three residues are residues in which a backbone C═O group is replaced with a backbone C═S group.
